(19) United States
(12) Patent Application Publication
(10) Pub. No.: US 2024/0374801 A1
(43) Pub. Date: Nov. 14, 2024

(54) Container for Peritoneal Dialysis, Corresponding Kit and Method for Calculating Ultrafiltration Volume

(71) Applicants: Fresenius Medical Care Deutschland GmbH, Bad Homburg (DE); Fresenius Medical Care Asia Pacific Ltd., Hong Kong (CN)

(72) Inventors: Peng Yuan, Bad Homburg (DE); Zhen Liu, Bad Homburg (DE)

(21) Appl. No.: 18/686,608

(22) PCT Filed: Aug. 31, 2021
(86) PCT No.: PCT/CN2021/115681

§ 371 Date: Feb. 26, 2024

(51) Int. Cl. A61M 1/28

(52) U.S. Cl. CPC A61M 1/285; A61M 2205/3306; A61M 2205/3334; A61M 2205/3393; A61M 2205/583

(57) ABSTRACT

Disclosed herein is a container for peritoneal dialysis, comprising an enclosure, configured to accommodate a fluid; an elastic tube, configured to be stretched for indicating a length change thereof according to the fluid flowing into and/or out of the enclosure; and at least one position mark, disposed on the enclosure or a rigid connector tube for the enclosure; wherein the position mark assists to determine the volume of the fluid flowing in and/or out of the enclosure. Also disclosed are a corresponding kit and methods for calculating the ultrafiltration volume. According to the disclosure, the ultrafiltration volume can be determined efficiently and conveniently, and can assist patents to tightly monitor their health and timely alert in case of a worse trend to be predicted.

### CROSS-REFERENCE TO RELATED APPLICATIONS

The present application is the national stage entry of International Patent Application No. PCT/CN2021/115681, filed on Aug. 31, 2021, the disclosure of which is incorporated herein by reference.

### TECHNICAL FIELD

The present disclosure relates to a container for peritoneal dialysis (PD), a corresponding kit and a method for calculating ultrafiltration volume.

### BACKGROUND ART

It is known that the peritoneal dialysis is one of most popular dialysis therapies carried out at home usually, which aims to remove toxic substances and metabolites normally removed by the kidneys, and to aid in regulation of fluid and electrolyte balance. The procedure is accomplished by infusing PD solution through a conduit into the peritoneal cavity of a patient, where the osmosis and diffusion occur across the peritoneal membrane between plasma of the patient and PD the solution.

A continuous ambulatory peritoneal dialysis (CAPD) and an automated peritoneal dialysis (APD) are two main types of PD therapy. CAPD allows the patient to exchange the PD solution 3 to 4 times per day, where a double-bag system is usually used in which one bag for containing the fresh PD solution and a separate empty bag for the patient's effluent (waste solution). The double-bag system provides a new solution bag and a new drainage bag for each PD treatment or exchange. In addition, CAPD can be performed by using a single-bag system, wherein only a solution bag is provided. The patient usually uses a used empty solution bag from a previous treatment or exchange as the drainage bag of a subsequent treatment or exchange.

When PD patients operate CAPD treatment, they need to make records for each treatment, including how much volume of fresh PS solution (so called influent) is introduced into the peritoneal cavity and how much volume of the effluent is drain out, and then work out the ultrafiltration volume for each individual solution exchange. Usually, PD patients can easily use scales to weigh the influent and effluent at home. However, if a PD patient wants to improve his/her mobility and lives in a more normal life, e.g. enable to go to work, go camping or hiking, even travel for a short vacation but a scale is not available on hand sometime, it may be difficult to precisely calculate the ultrafiltration volume and manage the treatment in a consistent way. On the other hand, even if the patient has a scale to weigh the fluid and also takes notes for the treatment by hand at the beginning, sooner or later, the patient may feel boring or reluctant to scale the fluid every time, but just write down an estimated value or even worst to keep a same value as the previous one, which will bring the patients, nurses and doctors into trouble to monitor the quality of the treatment and timely take necessary measure to maintain patient's health.

Some solutions are under development, e.g. by installing an individual flow meter, even an embedded flow sensor to determine an ultrafiltration volume based on the measured inflow volume and the outflow volume; however, it makes the relatively simple CAPD treatment more complicated in the view of technology, safety and user's operation: meanwhile, it also generates additional cost for each PD treatment.

Therefore, there is a need to provide a solution for patient's easy operation but still relative convenience to effectively and efficiently calculating the ultrafiltration volume to improve life quality and mobility.

### SUMMARY OF THE DISCLOSURE

In view of the problems existing in the prior art, an object of the disclosure is to provide a container for peritoneal dialysis, a corresponding kit and a method for calculating ultrafiltration volume.

For achieving this object, according to a first aspect, provided is a container for peritoneal dialysis, comprising an enclosure, configured to accommodate a fluid: an elastic tube, configured to be stretched for indicating a length change thereof according to the fluid flowing into and/or out of the enclosure; and at least one position mark, disposed on the enclosure or a rigid connector tube for the enclosure:

wherein the position mark assists to determine the volume of the fluid flowing in and/or out of the enclosure.

According to an optional embodiment of the present disclosure, the container further comprises at least two additional position marks, disposed on the elastic tube, and configured to indicate the length change of the elastic tube.

According to an optional embodiment of the present disclosure, said at least one position mark is disposed on the rigid connector tube, and only one additional position mark is disposed on the elastic tube for indicating a length change of the position mark relative to said at least one position mark.

According to an optional embodiment of the present disclosure, the at least one position mark is disposed on the rigid connector tube so that the highest edge of said at least one position mark is in line with the upper edge of the rigid connector tube; or the at least one position mark is disposed on the rigid connector tube so that the lowest edge of said at least one position mark is in line with the lower edge of the rigid connector tube.

According to an optional embodiment of the present disclosure, the length change of the elastic tube is identified by an image recognition technology; and images are taken, preferably by a smart phone, before and after the elastic tube is stretched.

According to an optional embodiment of the present disclosure, the container is a bag with a fresh solution, and the elastic tube is stretched for indicating a length reduction according to the fluid flowing out of the enclosure.

According to an optional embodiment of the present disclosure, the container is an empty bag for accommodating a waste fluid, and the elastic tube is stretched for indicating a length increase according to the fluid flowing into the enclosure.

According to an optional embodiment of the present disclosure, the elastic tube is a separate tube segment connected with the enclosure or the rigid connector tube.

According to an optional embodiment of the present disclosure, the elastic tube is a separate tube segment assembled with a fluid transfer tubing.

According to a second aspect, provided is an assembly comprising a kit comprising the container described above.

According to a third aspect, provided is a method for determining ultrafiltration volume by using the container described above or the kit described above. The method comprises: taking at least a first image, preferably by a smart phone, before a fluid is introduced into the enclosure: introducing the fluid into the enclosure; taking at least a second image after the elastic tube is stretched due to the introducing of the fluid into the enclosure: identifying a first length change of the elastic tube; and calculating the volume of the fluid or the ultrafiltration volume according to a function between the length change of the elastic tube and the weight of the fluid accommodated in the enclosure.

According to an optional embodiment of the present disclosure, if a fresh solution remains after the fresh solution is partially used, for example introduced into a patient's cavity, after calculating the volume of the fluid, the method further comprises introducing the remaining fresh solution into the enclosure; taking at least a third image after the elastic tube is stretched: identifying a second length change of the elastic tube; and calculating the volume of the remaining fresh solution or the volume of the fresh solution which is partially used.

According to an optional embodiment of the present disclosure, the method further comprises: introducing the fluid out of the enclosure before introducing the remaining fresh solution into the enclosure: or after calculating the volume of the fluid, introducing the remaining fresh solution into the enclosure without introducing the fluid out of the enclosure.

According to an optional embodiment of the present disclosure, the method further comprises taking at least a fourth image, preferably by a smart phone, before a fluid is introduced out of the enclosure; introducing the fluid out of the enclosure; taking at least a fifth image after the elastic tube is stretched: identifying a length change of the elastic tube; and calculating the volume of the remaining fresh solution or the volume of the fresh solution which is partially used.

According to an optional embodiment of the present disclosure, the relation between the length change of the elastic tube and the weight of the fluid accommodated in the enclosure is predetermined, preferred in a linear way.

According to the disclosure, the ultrafiltration volume of each PD treatment can be efficiently and conveniently calculated without making use of additional scale, furthermore, each ultrafiltration volume can be automatically obtained and stored in the App for patients to manage their treatment data, and display (or even predict) the trend of the quality of PD treatment during a certain period: which greatly facilitates patients' mobility and improves their living style. Additionally, the treatment records collected in such a complete manner will assist patents to closely monitor their health and timely or even in advance alert patients in case of a worse trend to be predicted: furthermore, such real records will be a valuable reference for a doctor or physician to supervise patients' each treatment, make medical recommendation and even decide a new prescription.

### BRIEF DESCRIPTION OF THE DRAWINGS

The disclosure and advantages thereof will be further understood by reading the following detailed description of some exemplary embodiments with reference to the drawings in which:

FIG. 1 shows a PD kit according to an exemplary embodiment of the present disclosure.

FIG. 2 shows a container with an elastic tube according to another exemplary embodiment of the present disclosure.

FIG. 3 shows the distance change between the two marks after the elastic tube is stretched according to an exemplary embodiment of the present disclosure.

FIG. 4 shows a flow chart of a method for determining ultrafiltration volume according to an exemplary embodiment of the present disclosure.

FIG. 5 shows a diagram of the strain property of the elastic tube according to an exemplary embodiment of the present disclosure.

FIG. 6 shows a further flow chart of a method for determining ultrafiltration volume according to an exemplary embodiment of the present disclosure.

FIG. 7 shows a further flow chart of a method for determining ultrafiltration volume according to an exemplary embodiment of the present disclosure.

FIG. 8 shows the position mark according to another exemplary embodiment of the present disclosure.

FIG. 9 shows the container according to another exemplary embodiment of the present disclosure.

FIG. 10 shows the container according to another exemplary embodiment of the present disclosure.

FIG. 11 shows one way for recording the length change of the elastic tube before and after the stretch according to the exemplary embodiment of the present disclosure as shown in FIG. 10.

FIG. 12 shows another way for recording the length change of the elastic tube before and after the stretch according to the exemplary embodiment of the present disclosure as shown in FIG. 10.

FIG. 13 shows another way for recording the length change of the elastic tube before and after the stretch according to the exemplary embodiment of the present disclosure as shown in FIG. 10.

FIG. 14 shows the container according to another exemplary embodiment of the present disclosure.

FIG. 15 shows one way for recording the length change of the elastic tube before and after the stretch according to the exemplary embodiment of the present disclosure as shown in FIG. 14.

FIG. 16 shows another way for recording the length change of the elastic tube before and after the stretch according to the exemplary embodiment of the present disclosure as shown in FIG. 14.

FIG. 17 shows another way for recording the length change of the elastic tube before and after the stretch according to the exemplary embodiment of the present disclosure as shown in FIG. 14.

### DETAILED DESCRIPTION OF PREFERRED EMBODIMENTS

Some exemplary embodiments of the present disclosure will be described hereinafter in more details with reference to the drawings to better understand the basic concept and advantages of the present disclosure.

According to the disclosure, herein firstly proposed is a container for peritoneal dialysis, comprising: an enclosure, configured to accommodate a fluid: an elastic tube, configured to be stretched for indicating a length change thereof according to the fluid flowing into and/or out of the enclosure; and at least one position mark, disposed on the enclosure or a rigid connector tube for the enclosure; wherein the position mark assists to determine the volume of the fluid flowing in and/or out of the enclosure.

The container may particularly be configured in a bag-like form. The container can be used to prepare a dialysis solution or an intermediate solution for preparing the peritoneal dialysis solution or used to accommodate a waste effluent after a peritoneal dialysis treatment.

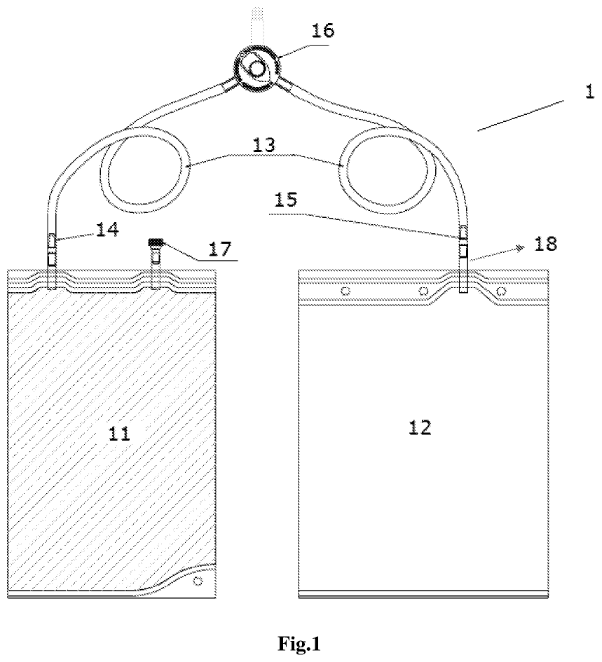

FIG. 1 shows such a PD kit according to an exemplary embodiment of the present disclosure.

As shown in FIG. 1, the PD kit 1 may comprise an enclosure 11 configured to accommodate a fresh solution: an empty enclosure 12 configure to accommodate a waste fluid; at least a transfer tubing 13 assembled on or in linking connectors 14 and/or 15 and configured to allow a fluid to flow into and/or out of the enclosures 11, 12 through a disc connector 16 according to the treatment operation: an injection port 17 optionally sealed in a sealing portion of the enclosures 11 and/or 12 for adding other liquid or sampling the fluid.

The skilled person in the art may understand that the PD kit 1 is shown in FIG. 1 in the most common usage status, and thus positional words, such as “above”, “below”, “top”, “bottom” and so on are used herein with respect to the most common usage status.

During the treatment operation, the effluent is firstly introduced out from a patient's peritoneal cavity into the empty enclosure 12, for example a drainage bag, through a channel built in the disc connector 16. Since the functions and operation on a disc are known by the skilled person in art, no further details will be described but the embodiment of the disc disclosed in the US filing (titled as multiway wave, US 2006/0005886 A1) can be incorporated herein as an example for better understanding. In some applications, the linking connector 15 disposed on the drainage bag 12 can be omitted.

According to an exemplary embodiment of the present disclosure, the new PD kit shown in FIG. 1 further comprises an elastic tube 18, which is connected to the drainage bag 12. Moreover, one or more marks are disposed on the elastic tube 18 and/or drainage bag 12 for recognizing the position changes as an example.

The skilled person in the art shall realize the embodiments illustrated in the drawings take the container with the drainage bag 12 as an example to further describe the details, but shall understand that the similar configuration of the container with the drainage bag 12 can be also applied on the container with the solution bag 11; therefore, the similar embodiments can be conceived based on the container with the solution bag 11. To make the disclosure clear and concise, most of embodiments will be illustrated in view of the drainage bag.

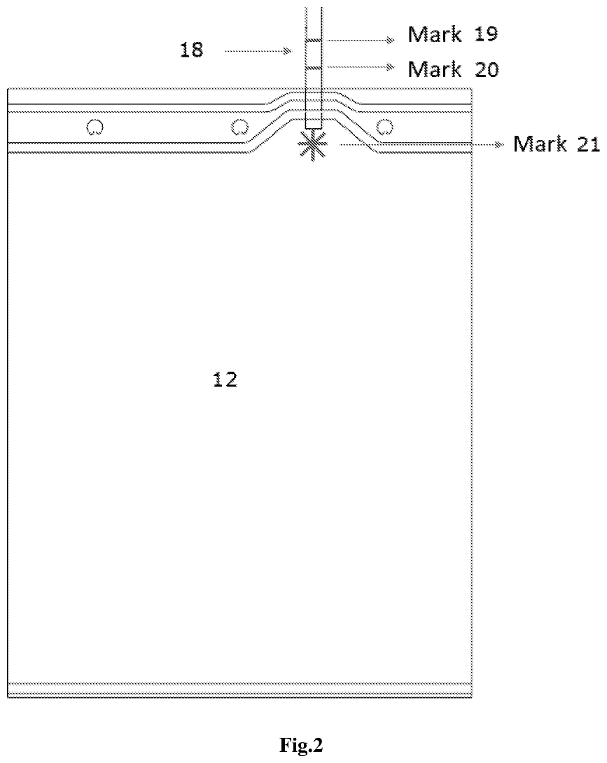

FIG. 2 shows the container with the elastic tube according to another exemplary embodiment of the present disclosure.

As shown in FIG. 2, at least three marks are disposed on the drainage bag and/or the elastic tube. In details, there are at least two marks 19, 20 disposed on the elastic tube 18 and at least one mark 21 disposed on the drainage bag 12, in which the mark 21 can be used as a position and/or calibration mark.

After the fresh solution is introduced and stored in a patient's peritoneal cavity for 3 to 4 hours, a next PD treatment can be performed by exchanging the fluid. The effluent is firstly drained out from the cavity and then introduced into the drainage bag 12. When the drainage bag 12 is hung on an IV pole or someone directly grasps an end of the transfer tubing 13 close to the elastic tube 18, due to the weight of the effluent in the drainage bag 12, the elastic tube 18 is stretched to some extent to generate a distance or length change between the two marks 19, 20.

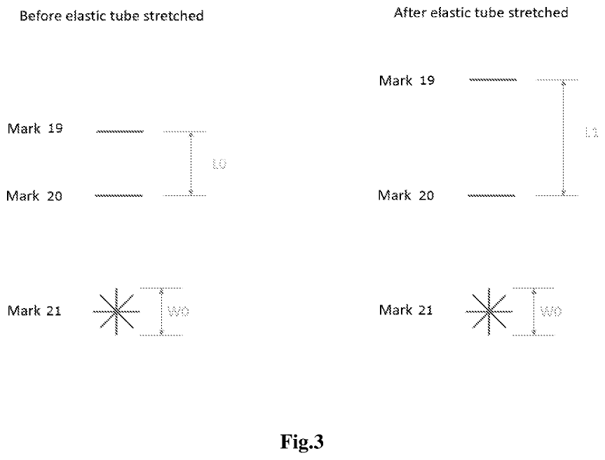

FIG. 3 shows the distance change between the two marks 19, 20 after the elastic tube is stretched according to an exemplary embodiment of the present disclosure. As shown in FIG. 3, the distance L0 between the two marks 19, 20 before the stretch has been prolonged to the new distance L1 after the stretch on the elastic tube 18, while the height of the mark 21, W0, is kept the same as before.

To automatically and efficiently recognize the distance change, a user or patient can use a smart phone to take pictures of the drainage bag 12 with the elastic tube 18 before and after the elastic tube 18 is stretched for comparison and analysis.

According to the disclosure, the ultrafiltration volume of each PD treatment can be efficiently calculated without making use of additional scale, furthermore, each ultrafiltration volume can be automatically obtained and stored in the App for patients to manage their treatment data, and display (or even predict) the trend of the quality of PD treatment during a certain period: which greatly facilitates patients' mobility and improves their living style. Additionally, the treatment records collected in such a complete manner will assist patents to tightly monitor their health and timely or even in advance alert patients in case of a worse trend to be predicted.

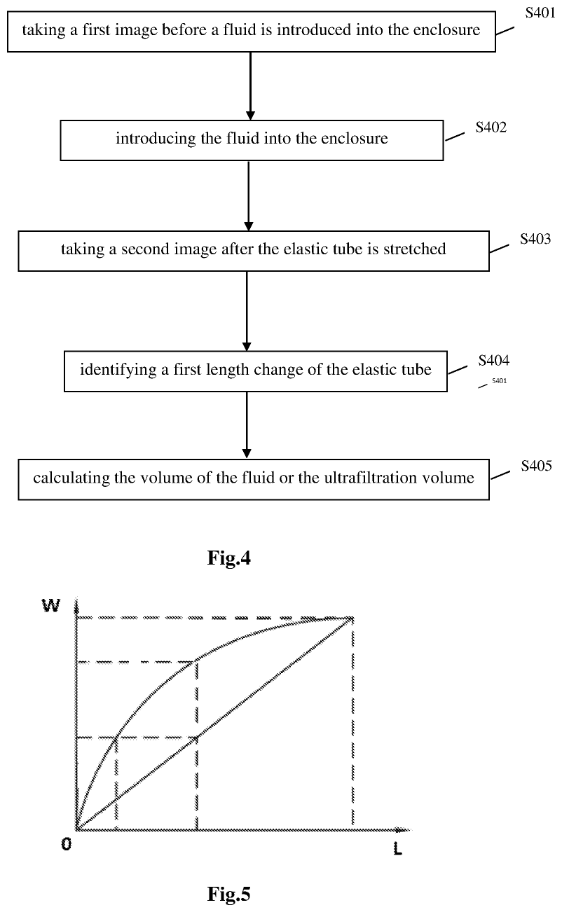

FIG. 4 shows a flow chart of a method for determining ultrafiltration volume according to an exemplary embodiment of the present disclosure.

As shown in FIG. 4, the method may comprise at least five steps described below.

A first step S401 for taking a first image before a fluid is introduced into the enclosure: preferably, before the effluent is introduced into the drainage bag 12, an initiate photo on the empty bag 12 with the elastic tube 18 is taken by a smart phone for recording the original positions of the marks 19, 20, 21. Because the stretch imposed by the weight of the empty bag 12 together with the elastic tube 18 could be nearly ignored before the treatment operation, the initiate photo can be easily taken, either in a vertical way (which means the drainage bag 12 can be hung on an IV pole or grasped by hand) or in a horizontal way (which means the drainage bag 12 will be horizontally put on a plate). As shown in FIG. 3, the mark 21 is used as a position mark: preferably, the height of the star mark 21 keeps W0 along the vertical direction during the operation. When the drainage bag 12 is empty without any fluid, the original distance between marks 19, 20 is L0.

A second step S402 for introducing the fluid into the enclosure: preferably, the effluent is firstly drained out from the cavity and simultaneously introduced into the drainage bag 12.

A third step S403 for taking a second image after the elastic tube is stretched due to the introducing of the fluid into the enclosure: preferably, after the drainage bag 12 is hung on an IV pole so that the elastic tube 18 is stretched to generate an obvious length change, at least a second photo on the bag 12 and/or the elastic tube 18 is taken by the smart phone for recording the current positions of the marks 19, 20, 21. Optionally, a few photos can be taken for the smart phone to automatically identify the highest quality one, which is recommended as the second photo for the comparison later.

As shown in FIG. 3, the mark 21 stays at the same position without a visible shape transformation, for example, the height of the star mark 21 almost keeps W0 along the vertical direction after the elastic tube 18 is stretched because of the weight of drainage bag with the effluent, while the distance between the marks 19, 20 is increased and prolonged from L0 to L1 accordingly.

A fourth step S404 for identifying a first length change of the elastic tube; preferably, after comparing the second photo with the initiate photo, the length change of the elastic tube 18 can be recognized by figuring out the distance change between the marks 19, 20. That's to say, the pixel distance between the marks 19, 20, PL1, and the pixel length of mark 21, PW0, can be calculated and identified by the smart phone with an application installed for identifying the new distance between marks 19, 20, L1, after the higher stretch.

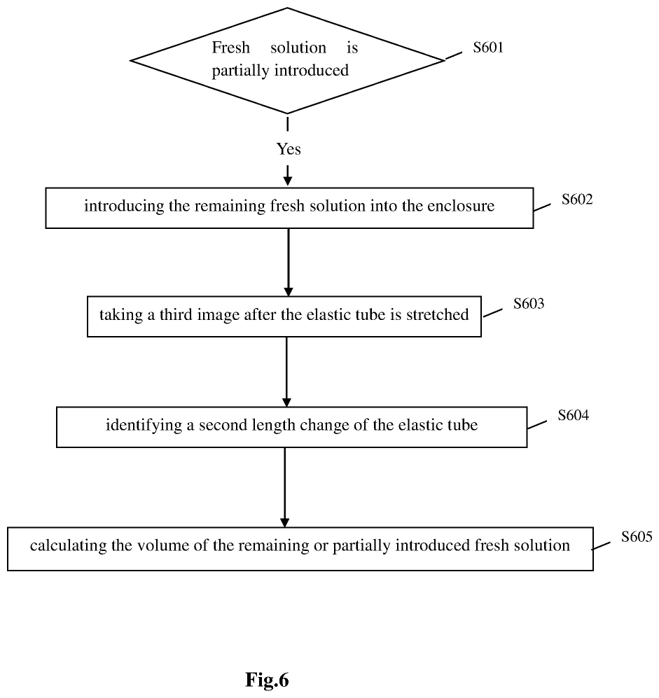

A fifth step S405 for calculating the volume of the fluid or the ultrafiltration volume according to a preset algorithm. In detail, the preset algorithm mainly relates to a function between the length change of the elastic tube and the weight of the fluid accommodated in the enclosure as explained in FIG. 5.

According to the method, the new PD container or PD Kit can be used to efficiently calculate the effluent exchange and then conveniently helps patients to determine the ultrafiltration volume of each PD treatment without making use of additional scale, which helps a lot to improve patients' mobility and may bring them back to a normal living style. Moreover, patients don't need to scale the fluid and manually take notes for recording the data by hand, alternatively, each ultrafiltration volume can be automatically obtained and stored in the App for patients to manage their treatment data, and display (or even predict) the trend of the quality of PD treatment during a certain period: which greatly releases patients or patients' helper (eg. the families, relatives or other helpers) from the boring daily work and beneficially assists to keep the records correct and complete.

FIG. 5 shows a diagram of the strain property of the elastic tube 18 according to an exemplary embodiment of the present disclosure.

As shown in FIG. 5, the strain property of the elastic tube aims to work in a similar manner as a spring scale, which can be determined by a function Y=f(L); where Y is the weight and L is a deformation of length. The function f(L) depends on the property of the material and the manufacture process for making the elastic tube, e.g. the material itself, a single layer or multiple layers, the length and/or diameter of the tube and thickness of the wall of the tube: which can be deduced from a tensile test in advance, then stored in the application for the comparison and calculation purpose. Preferably, it would be better to keep the elastic tube work in a linear way in the range of the normal effluent exchange volume (eg. no more than 5 liters, preferably no more than 3 liters); however, the skilled person in art shall understand, in principle, a non-linear function of f(L) is feasible and may be exploited in the disclosure as well, especially by controlling the non-linear function in a limited range of the fluid exchange volume (eg. no more than 3 liters, preferably no more than 2.5 liters). As a preferred embodiment, the weight of the effluent and the bag is linear with the distance change of from L0 to L1 for easy comparison and calculation.

Accordingly, the effluent volume, Veff, will be calculated based on the following equation set.

$$PW 0 / W 0 = PL 1 / L 1 \qquad ( 1 )$$

$$Wbag + Weff = f ( L 1 - L 0 ) \qquad ( 2 )$$

$$Veff = Weff / D \qquad ( 3 )$$

Normally, the weight, Wbag, of drainage bag and elastic tube depends on the manufacture and would be a constant value once the design is fixed; and the effluent volume, Veff, will be calculated based on the effluent weight, Weff, over the density of the fluid (eg. the effluent), D. For CAPD treatment, the density D of the fluid is approximately set at 1.0.

For the CAPD treatment that the whole solution in the enclosure 11, for example, a solution bag, is completely filled in a patient's peritoneal cavity, the influent volume, Vin, is always a fixed value depending on the manufacture's specification, for example, 2 liters. Then, the ultrafiltration volume, Veff−Vin, will be finally determined.

According to the disclosure, the ultrafiltration volume of each PD treatment can be efficiently calculated without making use of additional scale, furthermore, each ultrafiltration volume can be automatically obtained and stored in the App for patients to manage their treatment data, and display (or even predict) the trend of the quality of PD treatment during a certain period: which greatly facilitates patients' mobility and improves their living style. Moreover, patients don't need to scale the fluid and manually take notes for recording the data by hand; while each ultrafiltration volume can be automatically obtained and stored in the App for patients to manage their treatment data, and display (or even predict) the trend of the quality of PD treatment during a certain period: which greatly releases patients from the boring daily work and also assists to keep the records complete and correct.

FIG. 6 shows a further flow chart of a method for determining ultrafiltration volume according to an exemplary embodiment of the present disclosure.

According to another exemplary embodiment of the present disclosure, for the CAPD treatment that only a part of the solution in the solution bag 11 is filled in a patient's peritoneal cavity when a treatment is disturbed or the patient feels unwell for a complete filling, the influent volume herein named as Vinp shall be additionally determined before the actual ultrafiltration volume can be calculated by Veff−Vinp.

In one embodiment, the remaining fresh solution in the solution bag 11 can be figured out by means of the drainage bag 12 by the method described in FIG. 4. In detail, after the volume of the effluent, Veff, is determined, the method for determining ultrafiltration volume further comprises the following steps:

A step S601, whether the solution in the solution bag 11 is partially filled in a patient's peritoneal cavity or not? If yes, for example, a patient feels unwell and decides to partially fill in some volume of the fresh solution, or the volume of the fresh solution is roughly monitored by an additional means and the partial introduction of the solution is reminded when the introduced volume is obviously below the original volume in the solution bag 11: wherein the additional means can be a flow meter coupling to or integrated in transfer tubing 13 or catheter extension (not shown in FIG. 1).

A step S602, the remaining fresh solution will be further into the enclosure: in one of embodiments, the remaining fresh solution will be further into the drainage bag 12 through a fluid connection channel from the solution bag 11 to the drainage bag 12 via the transfer tubing 13 and the disc 16.

A step S603, at least a third image will be taken after the elastic tube is stretched for identifying a second length change of the elastic tube. Such photo can be taken in a similar manner described in step S403.

A step S604, a second length change of the elastic tube will be identified in a similar manner described in step S404.

A step S605, the volume of the remaining fresh solution or the partially introduced fresh solution will be calculated. Since the whole solution in the solution bag 11 is a fixed volume, once the volume of the remaining fresh solution is determined, the volume of the fresh solution partially introduced into the patient's cavity, Vinp, can be figured out accordingly.

Furthermore, to determine the partial influent volume, Vinp, there're two options which can be implemented by either (a) introducing the effluent out of the drainage bag 12 before introducing the remaining fresh solution into the enclosure: or (b) directly introducing the remaining fresh solution into the enclosure without draining the effluent out of the drainage bag 12 after the volume of the effluent is calculated. The former option (a) aims to make use of the empty drainage bag 12 to directly calculate the volume of the remaining fresh solution due to a new length change in relation to the weight of the remaining fresh solution: which means at least after the step S403, preferably, after the Veff being calculated in the step S405, the effluent will be completely drained out from the drainage bag 12 before starting the step S602: while the latter option (b) aims to calculate the total volume of the effluent together with the remaining fresh solution, then determines the volume of the remaining fresh solution by deducting the previously calculated effluent volume, Veff, and finally figures out the partial influent volume, Vinp.

According to the disclosure, the ultrafiltration volume of each PD treatment can be efficiently calculated without making use of additional scale, furthermore, each ultrafiltration volume can be automatically obtained and stored in the App for patients to manage their treatment data: which greatly facilitates patients' mobility and improves their living style. Additionally, the treatment records collected in such a complete manner will assist patents to closely monitor their health by displaying (or even predicting) the trend of the quality of PD treatment during a certain period, and timely (or even in advance) alert patients in case of a worse trend to be predicted: furthermore, such real records will be a valuable reference for a doctor or nurse to supervise patients' each treatment, make medical recommendation and even decide a new prescription.

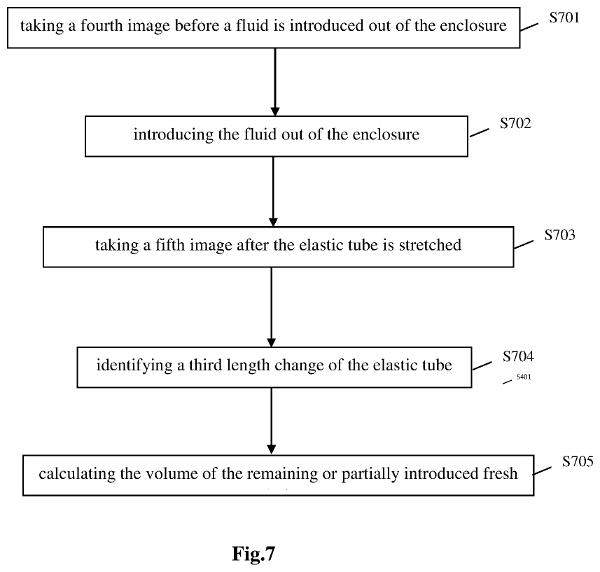

FIG. 7 shows a further flow chart of a method for determining ultrafiltration volume according to an exemplary embodiment of the present disclosure.

In an alternative embodiment, the solution bag 11 can be made in the same manner as the drainage bag 12 where an elastic tube 18 together with marks is disposed so that the volume of the partial influent volume, Vinp, of the fresh solution in the solution bag 11 can be calculated in a similar way. The skilled person in the art may understand that both the solution bag 11 and the drainage bag 12 are configured with such an elastic tube, therefore, the volume or weight of the liquid in either solution bag or drainage bag can be determined independently: therefore, the steps to calculate the volume of the partial influent volume, Vinp, of the fresh solution in the solution bag 11 and the steps to calculate the volume or weight of the effluent in the drainage bag 12 are not necessarily carried out one after the other and instead can be carried out in any suitable manner, for example, simultaneously or even overlapping in time.

In one option that the method for determining the volume of the effluent, Veff, is carried out as illustrated in FIG. 4: after the volume of the effluent, Veff, is determined, the method for determining ultrafiltration volume further comprises the following steps:

A step S701, taking at least a fourth image before a fluid is introduced out of the enclosure: preferably, before the fresh solution is introduced into a patient's cavity from the solution bag 11, at least a photo on the solution bag 11 with an elastic tube is taken by a smart phone for recording the original positions of the marks 19, 20, 21 when the solution bag 11 is hung on an IV pole or held by the patient. As indicated previously, the mark 21 is preferably used as a position mark which will stay at the same position without a visible shape transformation, for example, the height of the star mark 21 keeps W0 along the vertical direction during the operation. When the solution bag 12 with full solution is hung, the initiate distance between the marks 19, 20 is L0 with the weight of the accommodated solution imposed on the elastic tube.

A step S702, introducing the fluid out of the enclosure; preferably, the fresh solution is partially introduced out from the solution bag 11 and simultaneously introduced into a patient's cavity through the channel built by transfer tubing 13 and disc 16.

A step S703, taking at least a fifth image after the elastic tube is stretched; preferably, the elastic tube is stretched less than before due to the weight reduction because a part of fresh solution is drained out of the solution bag 11: where the distance between the marks 19, 20 is decreased and shortened from L0 to L1 accordingly. Hence, at least a new phone is taken by the smart phone for recording the current positions of the marks 19, 20, 21.

A step S704, identifying a third length change of the elastic tube: preferably, after comparing the fifth photo with the initiate photo of the solution bag 11, the length change of the elastic tube 18 can be recognized by figuring out the distance change between the marks 19, 20. That's to say, the pixel distance between the marks 19, 20, PL1, and the pixel length of the mark 21, PW0, can be calculated and identified by the smart phone with an application installed for identifying the new distance between the marks 19, 20, L1, after the less stretch.

A step S705, calculating the volume of the remaining fresh solution or the volume of the fresh solution partially introduced into the patient's cavity according to a preset algorithm. Similar as the calculation on the effluent volume, Veff described in FIG. 5, the partial influent volume, Vinp, of the fresh solution will be calculated based on the below equation set. Therefore, the ultrafiltration volume, Veff−Vinp, will be finally determined.

$$PW 0 / W 0 = PL 1 / L 1 \qquad ( 1 )$$

$$Winp = f ( L 0 - L 1 ) \qquad ( 2 )$$

$$Vinp = Winp / D \qquad ( 3 )$$

The skilled person in art shall understand, if the fourth image is taken in a way that the solution bag 11 isn't hung but disposed on a plate as an example, which means there's no gravity force disposed on the elastic tube while the fourth image is taken; and the original distance between marks 19, 20 is L0 without stretch imposed on the elastic tube. Under this embodiment, the weight of the fresh remaining solution, Winr, and the bag is linear with the distance change of from L0 to L1. Similar to the calculation on the effluent volume, Veff described in FIG. 5, the volume of the remaining fresh solution, Vinr, will be calculated based on the below equation set.

$$PW 0 / W 0 = PL 1 / L 1 \qquad ( 1 )$$

$$Wbag + Winr = f ( L 1 - L 0 ) \qquad ( 2 )$$

$$Vinr = Winr / D \qquad ( 3 )$$

Normally, the volume of the solution bag 11 is always a fixed value depending on the manufacture's specification, for example, 2 liters. Therefore, the partial influent volume, Vinp can be determined after the volume of the remaining fresh solution, Vinr, is directly figured out by the above equation set: then, the ultrafiltration volume, Veff-Vinp, will be finally determined.

The skilled person in the art may understand that these steps are not necessarily carried out in the above order and instead can be carried out in any suitable manner, for example, simultaneously or overlapping in time. Furthermore, the skilled person in art shall understand, the method for calculating ultrafiltration volume via smartphone can be implemented in a specific medical device or applied as one of functions integrated in an App installed in a smartphone, moreover the ultrafiltration volume by (Veff−Vin) per treatment can be automatically obtained and stored in the App for managing patient's treatment data, and display or even predict the trend or provide alerts in case of bad trend being predicted: further shared with patient's doctor for medical recommendations or reference for a new prescription.

According to the method, the new PD container or PD Kit can be used to efficiently calculate the effluent exchange and then conveniently helps patient to determine the ultrafiltration volume of each PD treatment without making use of additional scale, which greatly facilitates patients' mobility and improves their living style. Moreover, patients don't need to scale the fluid and take notes for recording the data by hand, while each ultrafiltration volume can be automatically obtained and stored in the App for patients to manage their treatment data, and display (or even predict) the trend of the quality of PD treatment during a certain period; which greatly releases patients from the boring work and also assists to keep the records complete and correct. Additionally, the treatment records collected in such a complete manner will assist patents to closely monitor their health by displaying (or even predicting) the trend of the quality of PD treatment during a certain period, and timely (or even in advance) alert patients in case of a worse trend to be predicted.

In the embodiments described above, the position mark 21 is disposed on the enclosure (either the drainage bag and/or the solution bag); while the skilled person in the art may understand that the location of the position mark 21 is not limited to the upper surface of the enclosure; any other locations may be feasible as well as long as it can keep the position mark undeformed or invisibly deformed when the elastic tube is stretched for recognizing the length change; which greatly provides more flexibility for manufacturing the PD bag or Kit instead substantially changes the normal manufacture process.

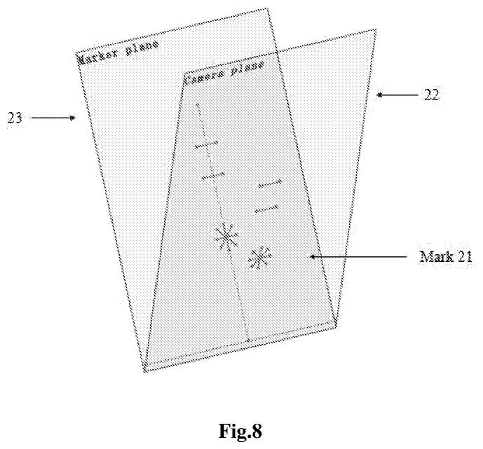

FIG. 8 shows the position mark 21 according to another exemplary embodiment of the present disclosure.

As shown in FIG. 8, the position mark 21 is further used to increase the accuracy of the recognition of a smart device (eg. a phone application). When a photo is taken but the application detects that Camera plane 22 is not parallel with the mark plane 23: which means the length of the mark 21 to be recorded will not be identical to that in the actual mark plane 23: the phone application will remind that a plane in parallel with the mark plane 23 shall be identified once again; or the angle between the camera plane 21 and the mark plane 23 will be calculated for adjusting the recorded length of the mark 21 to reflect the actual length.

Additionally, the type of the mark 21 is not limited to the one shown in the drawings, it is also possible to use gyroscope in a phone and the marks on a phone screen to locate the camera plane. Moreover, for a phone with more than one camera, it is also possible to use more cameras for locating the camera plane.

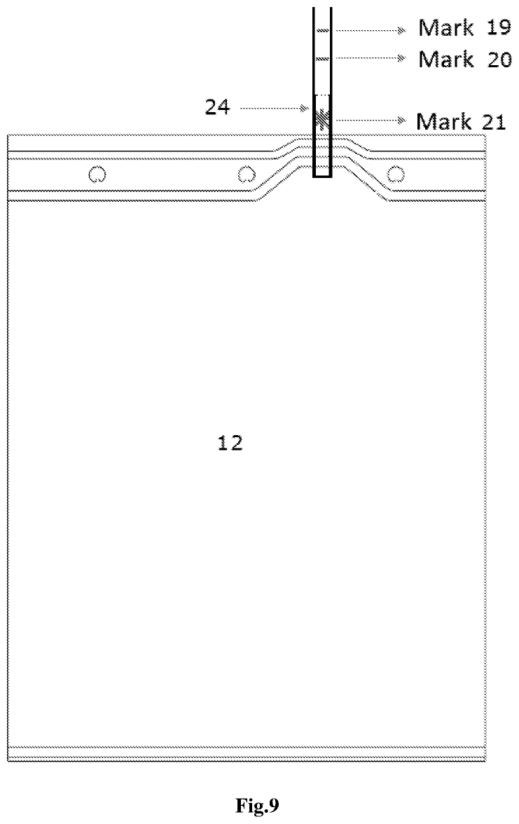

FIG. 9 shows the container according to another exemplary embodiment of the present disclosure.

As shown in FIG. 9, the position mark 21 is disposed on a connection tube 24 which was made from a nearly non-stretchable material in range of no less than 5 liters (preferably at least in the range of the normal effluent exchange) or a completely rigid material; where an elastic tube 18 is assembled on or in the connection tube 24 to connect each other: moreover, the marks 19 and 20 are still disposed on the elastic tube. Same as the embodiment described in FIG. 5, the effluent volume, Veff, will be calculated based on the below equation set accordingly.

$$PW 0 / W 0 = PL 1 / L 1 \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

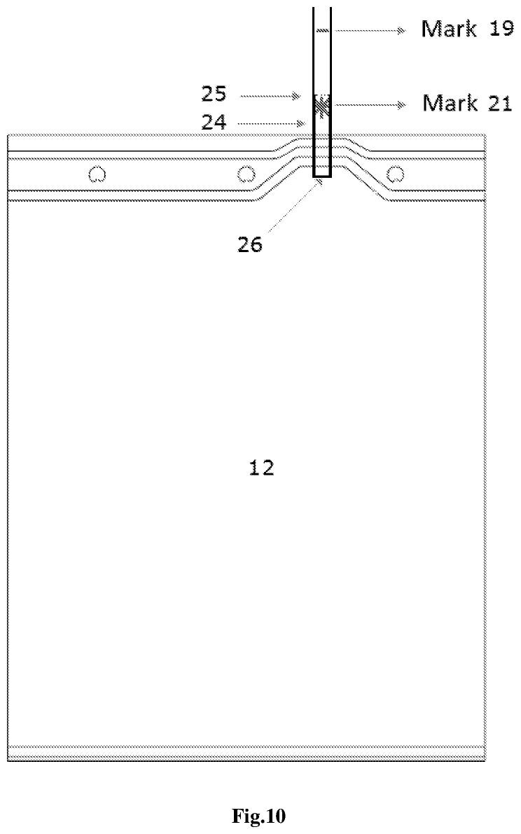

FIG. 10 shows the container according to another exemplary embodiment of the present disclosure.

As shown in FIG. 10, the position mark 21 is disposed on the nearly or completely rigid connection tube 24 which is similar as the embodiment in FIG. 9; however, there's only one mark (for example the mark 19) disposed on the elastic tube 18. Meanwhile, the position mark 21 is configured so that its highest edge aligns with the upper edge of the connection tube 24.

In the embodiments described above, the position mark 21 is disposed on connection tube instead of the enclosure, which can be realized as the position indicator during the connection tube manufacture; and also makes it possible to produce the PD bag without any process change.

FIG. 11 shows one way for recording the length change of the elastic tube before and after the stretch according to the exemplary embodiment of the present disclosure as shown in FIG. 10.

As shown in FIG. 11, L0 is the distance between mark 19 and the center of position mark 21 before the stretch. After the elastic tube is stretched, the distance between the mark 19 and the center of the position mark 21 is prolonged to L1: while the position mark 21 stays the same as before. Hence, the effluent volume, Veff, will be calculated based on the below equation set accordingly.

$$PW 0 / W 0 = ( PL 1 - PW 0 / 2 ) / ( L 1 - W 0 / 2 ) \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

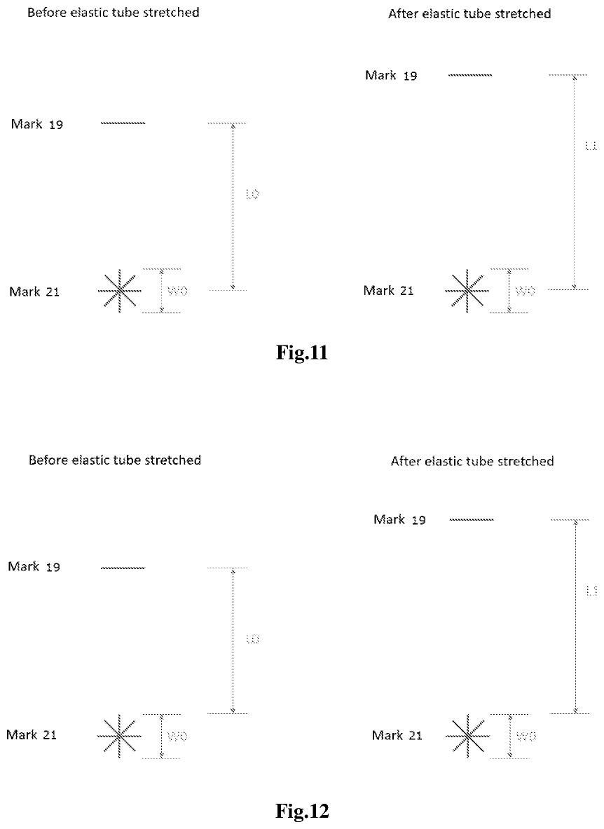

FIG. 12 shows another way for recording the length change of the elastic tube before and after the stretch according to the exemplary embodiment of the present disclosure as shown in FIG. 10.

As shown in FIG. 12, preferably, the position mark 21 is configured so that its highest edge aligns with the upper edge of the connection tube 24, and L0 is the distance from the mark 19 to the upper edge of the connection tube 24 (or the highest edge of the position mark 21) before the stretch. After the elastic tube is stretched, the distance between the mark 19 and the upper edge of the connection tube 24 is prolonged to L1; while the position mark 21 stays the same as before. Hence, the effluent volume, Veff, will be calculated based on the below equation set accordingly.

$$PW 0 / W 0 = PL 1 / L 1 \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

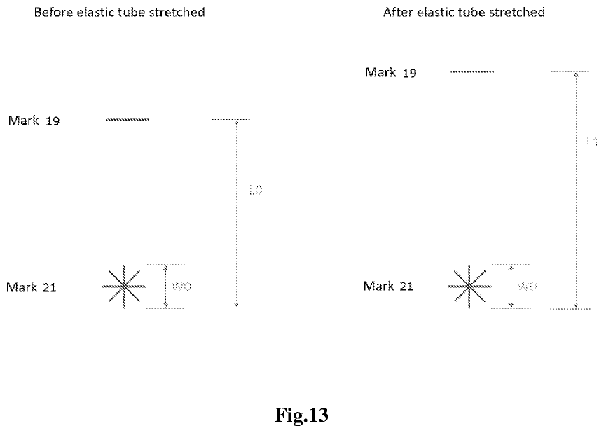

FIG. 13 shows another way for recording the length change of the elastic tube before and after the stretch according to the exemplary embodiment of the present disclosure as shown in FIG. 10.

Alternatively, as shown in FIG. 13, the position mark 21 is configured so that its highest edge aligns with the upper edge of the connection tube 24, and L0 is the distance from the mark 19 to the lowest edge of the position mark 21 before the stretch. After the elastic tube is stretched, the distance between the mark 19 and the lowest edge of the connection tube 24 is prolonged to L1; while the position mark 21 stays the same as before. Hence, the effluent volume, Veff, will be calculated based on the below equation set accordingly.

$$PW 0 / W 0 = ( PL 1 - PW 0 ) / ( L 1 - W 0 ) \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

According to the disclosure described above, the ultrafiltration volume of each PD treatment can be efficiently calculated without making use of additional scale, furthermore, each ultrafiltration volume can be automatically obtained and stored in the App for patients to manage their treatment data, and display (or even predict) the trend of the quality of PD treatment during a certain period: which greatly facilitates patients' mobility and improves their living style. Additionally, the treatment records collected in such a complete manner will assist patents to closely monitor their health and timely or even in advance alert patients in case of a worse trend to be predicted: furthermore, such real records will be a valuable reference for a doctor to monitor patients' each treatment, make timely recommendation and even decide a new prescription.

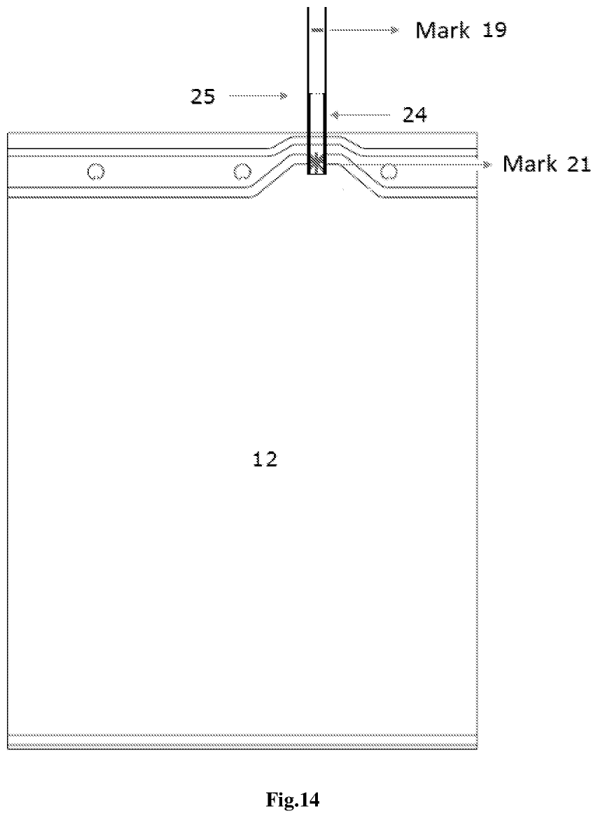

FIG. 14 shows the container according to another exemplary embodiment of the present disclosure.

As shown in FIG. 14, the position mark 21 is disposed on the nearly or completely rigid connection tube 24 which is similar to the embodiment in FIG. 9; however, there's only one mark (for example the mark 19) disposed on the elastic tube 18. Meanwhile, the position mark 21 is configured so that its lowest edge aligns with the lower edge of the connection tube 24. Moreover, the connection tube 24 has a standard length Lf, depending on the actual manufacture; and PLf is the pixel length of the connection tube 24.

In the embodiments described above, the position mark 21 is disposed on connection tube instead of the enclosure, which can be realized as the position indicator during the connection tube manufacture; and also makes it possible to produce the PD bag without any process change.

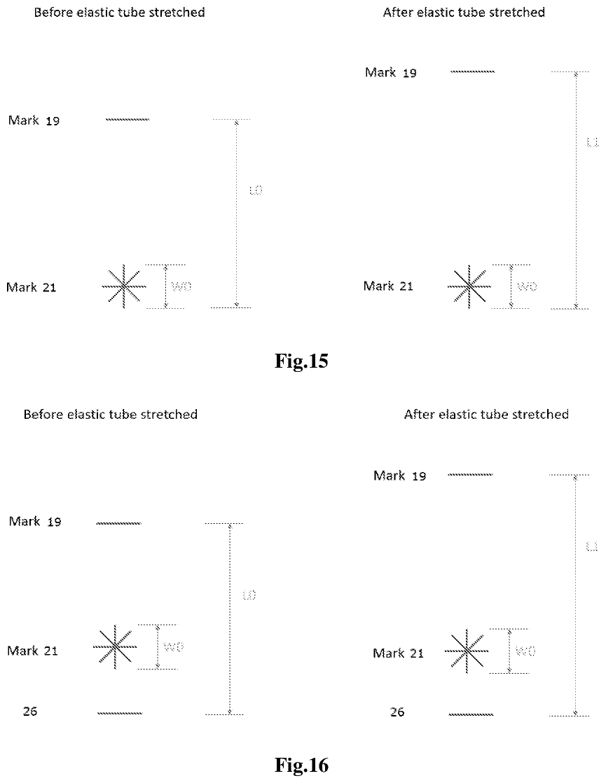

FIG. 15 shows one way for recording the length change of the elastic tube before and after the stretch according to the exemplary embodiment of the present disclosure as shown in FIG. 14.

As shown in FIG. 15, the position mark 21 is configured so that its lowest edge aligns with the lower edge of the connection tube 24, and L0 could be the distance from the mark 19 to the lower edge of the connection tube 24 before the stretch. After elastic tube 18 is stretched, the distance between the mark 19 and the lower edge of the connection tube 24 is prolonged to L1: while the position mark 21 stays the same as before. Hence, the effluent volume, Veff, will be calculated based on the below equation set accordingly.

$$PW 0 / W 0 = ( PL 1 - PLf ) / ( L 1 - Lf ) \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

FIG. 16 shows another way for recording the length change of the elastic tube before and after the stretch according to the exemplary embodiment of the present disclosure as shown in FIG. 14.

In one alternative embodiment shown in FIG. 16, the position mark 21 can be disposed anywhere of the nearly or completely rigid connection tube 24 as long as the connection tube 24 has a standard length Lf depending on the actual manufacture; which means the position mark 21 is in between the mark 19 and the lower edge 26 of the connection tube 24. L0 could be the distance from the mark 19 to the lower edge 26 of the connection tube 24 before the stretch. PLf is the pixel length of the connection tube 24. After the elastic tube is stretched, the distance between the mark 19 and the lower edge 26 of the connection tube is prolonged to L1. Therefore, the effluent volume, Veff, will be calculated based on the below equation set accordingly.

$$PW 0 / W 0 = ( PL 1 - PLf ) / ( L 1 - Lf ) \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

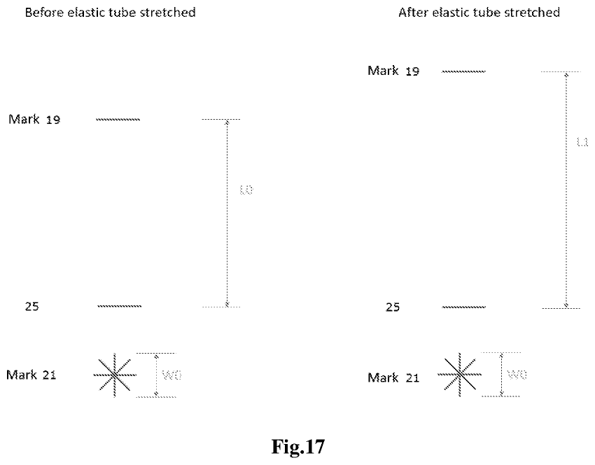

FIG. 17 shows another way for recording the length change of the elastic tube before and after the stretch according to the exemplary embodiment of the present disclosure as shown in FIG. 14.

In another alternative embodiment shown in FIG. 17, the position mark 21 can be disposed anywhere of the nearly or completely rigid connection tube 24 as long as the connection tube 24 has a standard length Lf depending on the actual manufacture; which means the position mark 21 is under the upper edge 25 of the connection tube 24. L0 could be the distance from the mark 19 to the upper edge 25 of the connection tube 24 before the stretch. PLf is the pixel length of the connection tube 24. After the elastic tube 18 is stretched, the distance between the mark 19 and the upper edge 26 of the connection tube 24 is prolonged to L1. Therefore, the effluent volume, Veff, will be calculated based on the below equation set accordingly.

$$PW 0 / W 0 = PL 1 / L 1 \qquad ( 1 )$$

$$Wbag + Weff = f ( L 1 - L 0 ) \qquad ( 2 )$$

$$Veff = Weff / D \qquad ( 3 )$$

Eventually, the ultrafiltration volume, (Veff−Vin) or (Veff−Vinp), will be finally determined by referring to the previous embodiments how to calculate the Vin or Vinp.

According to the disclosure described above, the ultrafiltration volume of each PD treatment can be efficiently calculated without making use of additional scale, furthermore, each ultrafiltration volume can be automatically obtained and stored in the App for patients to manage their treatment data, and display (or even predict) the trend of the quality of PD treatment during a certain period: which greatly facilitates patients' mobility and improves their living style. Additionally, the treatment records collected in such a complete manner will assist patents to tightly monitor their health and timely or even in advance alert patients in case of a worse trend to be predicted: furthermore, such real records will be a valuable reference for a doctor to supervise patients' each treatment, make medical recommendations and even decide a new prescription.

Alternatively, it is not necessary to print the marks on the PD container or PD kit, because the app or smart phone may be used to recognize some features on the PD kit, for example, injection tube, injection port, the words on bag or tubing, etc. Such features may be used as the marks for example the position mark 21. In other words, the concept disclosed herein can be realized but not limited to the printed marks; in case some features on PD kit which can be recognized by phone application is also feasible to be used as position indicators.

While certain embodiments have been described, these embodiments have been presented by way of example only and are not intended to limit the scope of the disclosure. The attached claims and their equivalents are intended to cover all the modifications, substitutions and changes as would fall within the scope and spirit of the disclosure.

1.-15. (canceled)

16. A container for peritoneal dialysis, comprising:
an enclosure, configured to accommodate a fluid;
an elastic tube, configured to be stretched for indicating a length change thereof according to the fluid flowing into and/or out of the enclosure; and
at least one position mark, disposed on the enclosure or a rigid connector tube for the enclosure;
wherein the position mark assists to determine the volume of the fluid flowing in and/or out of the enclosure.

17. The container according to claim 16, wherein the container further comprises:
at least two additional position marks, disposed on the elastic tube, and configured to indicate the length change of the elastic tube.

18. The container according to claim 16, wherein
said at least one position mark is disposed on the rigid connector tube, and
only one additional position mark is disposed on the elastic tube for indicating a length change of the position mark relative to said at least one position mark.

19. The container according to claim 18, wherein
said at least one position mark is disposed on the rigid connector tube so that the highest edge of said at least one position mark is in line with the upper edge of the rigid connector tube; or
said at least one position mark is disposed on the rigid connector tube so that the lowest edge of said at least one position mark is in line with the lower edge of the rigid connector tube.

20. The container according to claim 16, wherein
the length change of the elastic tube is identified by an image recognition technology; and
images are taken, preferably by a smart phone, before and after the elastic tube is stretched.

21. The container according to claim 16, wherein
the container is a bag with a fresh solution, and
the elastic tube is stretched for indicating a length reduction according to the fluid flowing out of the enclosure.

22. The container according to claim 16, wherein
the container is an empty bag for accommodating a waste fluid, and
the elastic tube is stretched for indicating a length increase according to the fluid flowing into the enclosure.

23. The container according to claim 16, wherein
the elastic tube is a separate tube segment connected with the enclosure or the rigid connector tube.

24. The container according to claim 16, wherein
the elastic tube is a separate tube segment assembled with a fluid transfer tubing.

25. The container according to claim 17, wherein
the length change of the elastic tube is identified by an image recognition technology; and
images are taken, preferably by a smart phone, before and after the elastic tube is stretched.

26. The container according to claim 18, wherein
the length change of the elastic tube is identified by an image recognition technology; and
images are taken, preferably by a smart phone, before and after the elastic tube is stretched.

27. The container according to claim 19, wherein
the length change of the elastic tube is identified by an image recognition technology; and
images are taken, preferably by a smart phone, before and after the elastic tube is stretched.

28. The container according to claim 17, wherein
the container is a bag with a fresh solution, and
the elastic tube is stretched for indicating a length reduction according to the fluid flowing out of the enclosure.

29. The container according to claim 18, wherein
the container is a bag with a fresh solution, and
the elastic tube is stretched for indicating a length reduction according to the fluid flowing out of the enclosure.

30. A kit comprising the container according to claim 16.

31. A method for determining ultrafiltration volume by using the container according to claim 16, the method comprising:
taking at least a first image, preferably by a smart phone, before a fluid is introduced into the enclosure;
introducing the fluid into the enclosure;
taking at least a second image after the elastic tube is stretched due to the introducing of the fluid into the enclosure;
identifying a first length change of the elastic tube; and
calculating the volume of the fluid or the ultrafiltration volume according to a function between the length change of the elastic tube and the weight of the fluid accommodated in the enclosure.

32. The method according to claim 31, wherein
if a fresh solution remains after the fresh solution is partially used, after calculating the volume of the fluid, the method further comprising
introducing the remaining fresh solution into the enclosure;
taking at least a third image after the elastic tube is stretched;
identifying a second length change of the elastic tube; and
calculating the volume of the remaining fresh solution or the volume of the fresh solution which is partially used.

33. The method according to claim 32, wherein the method further comprises:
introducing the fluid out of the enclosure before introducing the remaining fresh solution into the enclosure; or
after calculating the volume of the fluid, introducing the remaining fresh solution into the enclosure without introducing the fluid out of the enclosure.

34. The method according to claim 31, wherein the method further comprises
taking at least a fourth image, preferably by a smart phone, before a fluid is introduced out of the enclosure;
introducing the fluid out of the enclosure;
taking at least a fifth image after the elastic tube is stretched;
identifying a length change of the elastic tube; and
calculating the volume of the remaining fresh solution or the volume of the fresh solution which is partially used.

35. The method according to claim 31, wherein
the function between the length change of the clastic tube and the weight of the fluid accommodated in the enclosure is predetermined, preferred in a linear manner.
